(19) United States
(12) Patent Application Publication
(10) Pub. No.: US 2026/0318908 A1
(43) Pub. Date: Oct. 15, 2026

(54) TEST KIT

(71) Applicant: CMIC HOLDINGS Co., Ltd., Tokyo (JP)

(72) Inventor: Gakuho FUKUSHI, Tokyo (JP)

(21) Appl. No.: 19/297,459

(22) Filed: Aug. 12, 2025

(30) Foreign Application Priority Data
Aug. 13, 2024 (JP) 2024-134806

(51) Int. Cl. G01N 33/543; G01N 33/569; G06K 19/06

(52) U.S. Cl. CPC G01N 33/54387; G01N 33/56983; G06K 19/06028; G01N 2333/11; G01N 2333/165; G01N 2469/10

(57) ABSTRACT

A test kit 1 includes: a specimen dripping section 2 where a specimen is dripped; a labeled-antibody encapsulation layer 3 that contains a labeled antibody to be mixed with the specimen dripped onto the specimen dripping section; a migration layer 4 in which the specimen mixed with the labeled antibody in the labeled-antibody encapsulation layer 3 migrates; a control region 5 provided in the migration layer 4, in which an antibody that binds to the labeled antibody is disposed; and a test region 6 provided in the migration layer 4, in which an antibody that binds to an antigen to be tested is disposed. The control region 5 and/or the test region 6 are configured to display characters, numbers, or codes.

### BACKGROUND OF THE INVENTION

### Field of the Invention

The present invention relates to a test kit employing an immunochromatographic method.

### Description of the Related Art

Conventionally, test kits using immunochromatography have been used to test for influenza (acute influenza virus infection), the novel coronavirus disease, and the like. Conventional immunochromatography test kits are configured so that only a control line exhibits a color reaction when a virus to be tested is not detected but both the control line and a test line exhibit color reactions when the virus to be tested is detected. Therefore, a user can determine whether or not the virus to be tested has been detected by the presence or absence of a color reaction on the test line (for example, refer to Japanese Patent Laid-Open No. 2022-115777).

### SUMMARY OF THE INVENTION

However, conventional immunochromatography test kits have a problem in that they can only express information of “0” or “1”, representing the presence or absence of test lines as test results, and therefore cannot express complex information intended for users of the test kit, laboratory personnel, and others.

The present invention has been made in consideration of the problem described above and provides a test kit capable of expressing complex information intended for users of the test kit, laboratory personnel, and others as a test result.

A test kit according to the present invention is a test kit employing an immunochromatographic method, the test kit comprising: a specimen dripping section where a specimen is dripped; a labeled-antibody encapsulation layer that contains a labeled antibody to be mixed with the specimen dripped onto the specimen dripping section; a migration layer in which the specimen mixed with the labeled antibody in the labeled-antibody encapsulation layer migrates; a first test result display region (also referred to as a control region) provided in the migration layer, in which an antibody that binds to a labeled antibody or other substance indicative of a control state is disposed; and a second test result display region (also referred to as a test region) provided in the migration layer, in which an antibody that binds to a target component to be tested is disposed, wherein the control region and/or the test region are configured to display characters, numbers, or codes.

According to this configuration, in a test kit employing an immunochromatographic method, characters, numbers, or codes can be displayed by the control region and/or the test region. Therefore, it will be possible to display complex information (information having a larger information content than that of “0” or “1”, representing the presence or absence of test lines) intended for users of the test kit, laboratory personnel, and others as test results.

In the test kit according to the present invention, the control region and the test region are arranged to overlap each other, and their combination may display characters, numbers, or codes different from those shown in either region individually.

According to this configuration, the control region and the test region are arranged to overlap each other. Accordingly, it is possible to display more information through the combination of these regions. In this case, characters or the like (characters, numbers, or codes) different from those shown in either region individually can be displayed by their combination. In other words, the control region and the test region can each display information independently, while additional information can be displayed through their combined display.

In addition, in the test kit according to the present invention, the code may be a barcode that encodes predetermined information.

According to this configuration, a barcode that encodes predetermined information can be expressed by the code formed by the control region and/or the test region.

According to the present invention, complex information intended for users, laboratory personnel, and others can be displayed as a test result.

### BRIEF DESCRIPTION OF THE DRAWINGS

FIG. 1 is an explanatory diagram illustrating the principle of a test kit according to an embodiment of the present invention;

FIGS. 2A-2B are diagrams illustrating examples of the test kit according to the embodiment;

FIGS. 3A-3B are diagrams illustrating examples of the test kit according to the embodiment;

FIGS. 4A-4B are diagrams illustrating examples of the test kit according to the embodiment;

FIGS. 5A-5B are diagrams illustrating examples of the test kit according to the embodiment;

FIGS. 6A-6B are diagrams illustrating examples of the test kit according to the embodiment; and

FIGS. 7A-7B are diagrams illustrating examples of the test kit according to the embodiment.

### DETAILED DESCRIPTION OF THE INVENTION

Hereinafter, a test kit according to an embodiment of the present invention will be described with reference to the drawings. In this embodiment, an example is given of a test kit used for antigen testing of influenza viruses, novel coronaviruses, or the like.

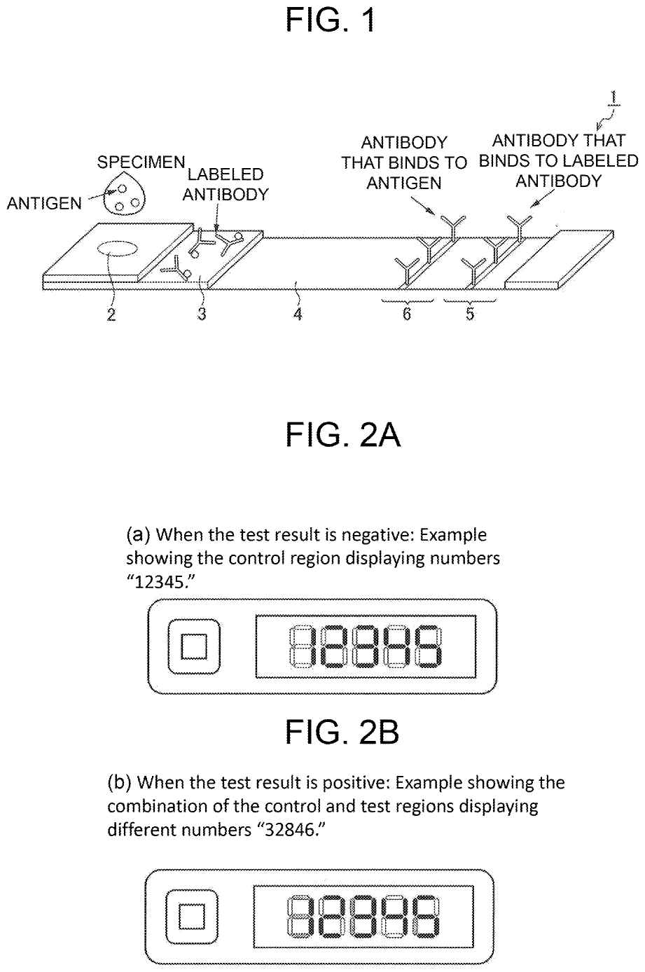

The configuration of the test kit according to this embodiment will be described with reference to the drawings. FIG. 1 is an explanatory diagram illustrating the principle of the test kit. As shown in FIG. 1, a test kit 1 according to this embodiment includes: a specimen dripping section 2 where a specimen is dripped; a labeled-antibody encapsulation layer 3 that contains a labeled antibody to be mixed with the specimen dripped onto the specimen dripping section; and a migration layer 4 in which the specimen mixed with the labeled antibody in the labeled-antibody encapsulation layer 3 migrates.

The migration layer 4 is further provided with a control region 5, in which an antibody that binds to the labeled antibody is disposed, and a test region 6, in which an antibody that binds to an antigen to be tested is disposed.

FIGS. 2A and 2B are diagrams illustrating examples of the test kit 1 according to this embodiment. In the examples of the test kit 1 shown in FIGS. 2A and 2B, the control region 5 is configured to display the number “12345” (see FIG. 2A). In this case, the control region 5 and the test region 6 are arranged to overlap each other, and their combination is configured to display the number “32846”, which differs from the number displayed by the control region 5 (see FIG. 2B). In this case, the test result can be displayed in a format that is not easily recognizable by third parties. By displaying test results in a manner that is difficult for third parties to interpret, privacy can be protected.

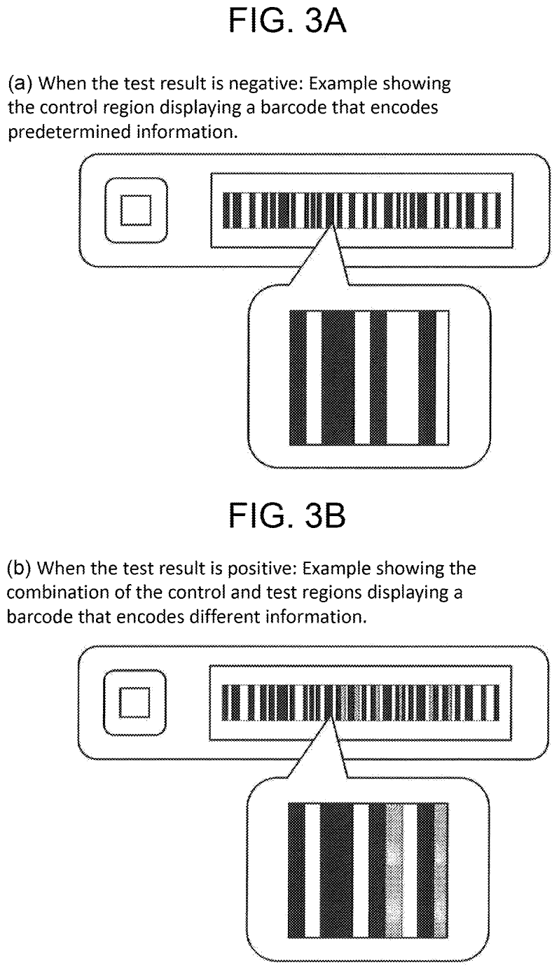

FIGS. 3A and 3B are diagrams illustrating other examples of the test kit 1 according to this embodiment. In the examples of the test kit 1 shown in FIGS. 3A and 3B, the control region 5 is configured to display a code (a barcode that encodes predetermined information) (see FIG. 3A). In this case, the control region 5 and the test region 6 are arranged to overlap each other, and their combination is configured to display a code that differs from that of the control region 5 (a barcode that encodes information different from that shown in FIG. 3A) (see FIG. 3B). In this case, the test result can be displayed in a format that is not easily recognizable by third parties. By displaying test results in a manner that is difficult for third parties to interpret, privacy can be protected.

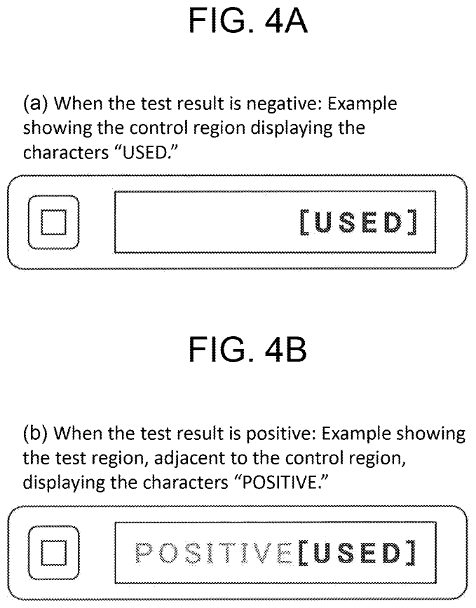

FIGS. 4A and 4B are diagrams illustrating other examples of the test kit 1 according to this embodiment. In the examples of the test kit shown 1 in FIGS. 4A and 4B, the control region 5 is configured to display the characters “USED” (see FIG. 4A). In this case, the test region 6 is provided adjacent to the control region 5, and the test region 6 is configured to display the characters “POSITIVE”, which differ from those of the control region 5 (see FIG. 4B).

In this case, the test result can be displayed in a format that is easily recognizable by third parties. By displaying “USED” and “POSITIVE” in an easy-to-understand manner as described above, positive or negative results that were difficult to recognize when shown by a simple line can be prevented from being misunderstood.

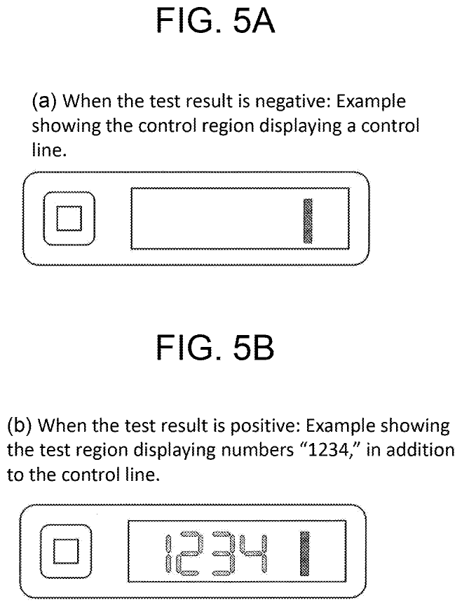

FIGS. 5A and 5B are diagrams illustrating other examples of the test kit 1 according to this embodiment. In the examples of the test kit 1 shown in FIGS. 5A and 5B, the control region 5 is configured to display a control line (see FIG. 5A). In this case, the test region 6 is configured to display the number “1234” (see FIG. 5B). In this case, the test result can be displayed in a format that is not easily recognizable by third parties. By displaying test results in a manner that is difficult for third parties to interpret, privacy can be protected.

According to the test kit 1 of this embodiment configured as described above, in the test kit 1 using immunochromatography, characters, numbers, or codes can be displayed by the control region 5 and/or the test region 6 (see FIGS. 2 to 5). Therefore, complex information—that is, information having a larger information content than “0” or “1” representing the presence or absence of test lines—can be displayed as test results for users of the test kit 1, laboratory personnel, and others.

In addition, in this embodiment, the control region 5 and the test region 6 are arranged to overlap each other. Accordingly, it is possible to display more information by the combination of the control region 5 and the test region 6 (see FIGS. 2 and 3). In this case, characters or the like (characters, numbers, or codes) different from those displayed by either the control region 5 or the test region 6 can be displayed through their combination. In other words, the control region 5 and the test region 6 can each display information independently, while additional information can be displayed through their combination.

In addition, in this embodiment, a barcode that encodes predetermined information can be displayed by a code formed by the control region 5 and/or the test region 6 (see FIGS. 3A and 3B).

While an embodiment of the present invention has been described by way of example, it should be understood that the scope of the invention is not limited to this embodiment, and various changes and modifications may be made according to the intended use within the scope of the claims.

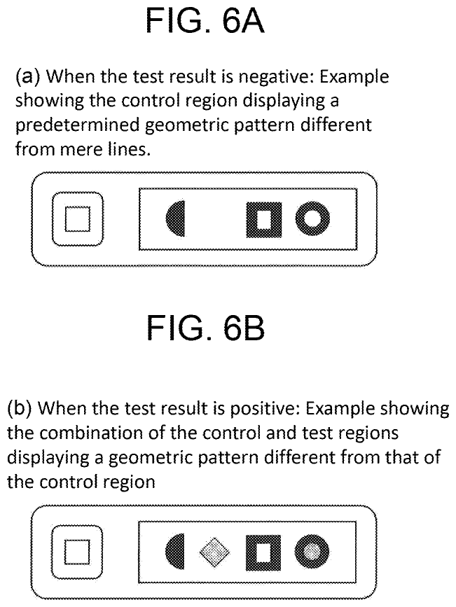

FIGS. 6A and 6B are diagrams illustrating other examples of the test kit 1 according to this embodiment. In the examples of the test kit 1 shown in FIGS. 6A and 6B, the control region is configured to display a predetermined geometric pattern (a pattern different from mere lines) (see FIG. 6A). In this case, the control region and the test region are arranged to overlap each other, and their combination is configured to display a geometric pattern (a pattern different from mere lines) that differs from that of the control region (see FIG. 6B).

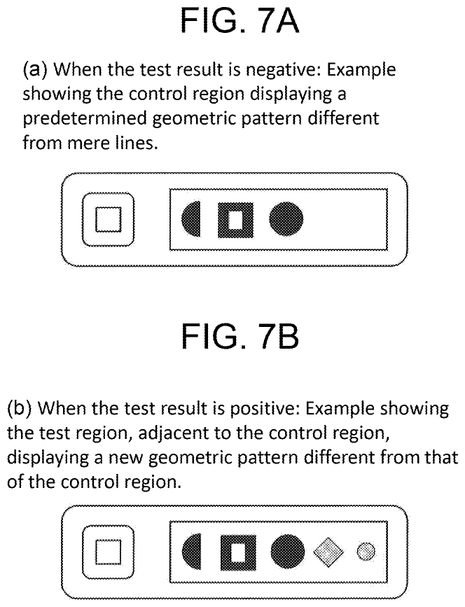

FIGS. 7A and 7B are diagrams illustrating other examples of a test kit 1 according to this embodiment. In the examples of the test kit 1 shown in FIGS. 7A and 7B, the control region is configured to display a predetermined geometric pattern (a pattern different from mere lines) (see FIG. 7A). In this case, the test region is provided adjacent to the control region, and the test region is configured to display a new geometric pattern (a geometric pattern different from the control region, which is a pattern different from mere lines) (see FIG. 7B).

Even in the test kits shown in FIGS. 6 and 7, complex information—that is, information having a larger information content than “0” or “1” representing the presence or absence of test lines—can be displayed as test results for users of the test kits, laboratory personnel, and others.

As described above, the test kit according to the present invention provides the advantageous effect of being able to display complex information—intended for users of the test kit, laboratory personnel, and others—as test results, and is useful as a test kit for antigen testing of influenza viruses and novel coronaviruses.

### REFERENCE SIGNS LIST

1 test kit
2 specimen dripping section
3 labeled-antibody encapsulation layer
4 migration layer
5 control region
6 test region

1. A test kit employing an immunochromatographic method, the test kit comprising:
a specimen dripping section where a specimen is dripped;
a labeled-antibody encapsulation layer that contains a labeled antibody to be mixed with the specimen dripped onto the specimen dripping section;
a migration layer in which the specimen mixed with the labeled antibody in the labeled-antibody encapsulation layer migrates;
a control region provided in the migration layer, in which an antibody that binds to a labeled antibody or other substance indicative of a control state is disposed; and
a test region provided in the migration layer, in which an antibody that binds to a target component to be tested is disposed, wherein
the control region and/or the test region are configured to display characters, numbers, or codes.

2. The test kit according to claim 1, wherein the control region and the test region are arranged to overlap each other, and
their combination displays characters, numbers, or codes different from those shown in either region individually.

3. The test kit according to claim 2, wherein the code is a barcode that encodes predetermined information.
